(19) United States
(12) Patent Application Publication
(10) Pub. No.: US 2023/0158281 A1
(43) Pub. Date: May 25, 2023

(54) SYSTEMS, DEVICES, AND METHODS FOR DELIVERING A SUBSTANCE WITHIN A TARGET TISSUE

(71) Applicant: The Regents of the University of California, Oakland, CA (US)

(72) Inventors: David Conrad, San Francisco, CA (US); Adrian E. House, San Francisco, CA (US); Matthew Jones, San Francisco, CA (US); Andrew N. Goldberg, San Francisco, CA (US); Nicole Jiam, San Francisco, CA (US)

(21) Appl. No.: 17/918,064

(22) PCT Filed: Apr. 10, 2021
(86) PCT No.: PCT/US2021/026753

§ 371 Date: Oct. 10, 2022

Related U.S. Application Data
(60) Provisional application No. 63/007,995, filed on Apr. 10, 2020.

(51) Int. Cl. A61M 31/00; A61M 37/00; A61K 31/573

(52) U.S. Cl. CPC A61M 31/002; A61M 37/0069; A61K 31/573; A61M 2210/0618; A61M 2205/04

(57) ABSTRACT

In one embodiment, an implant includes a body having a distal end and a proximal end, the distal end including a sharp pointed tip that is configured to pierce and cut through tissue, an anchoring element extending outward from the body configured to prevent migration of the implant within tissue in which the implant has been implanted, and a therapeutic substance that is slowly released by the implant into the tissue over time, wherein the implant is made of a biocompatible and bioabsorbable material.

### CROSS-REFERENCE TO RELATED APPLICATION

This application claims priority to co-pending U.S. Provisional Application Ser. No. 63/007,995, filed Apr. 10, 2020, which is hereby incorporated by reference herein in its entirety.

### BACKGROUND

There are a variety of health conditions that are treated using invasive surgical procedures. One such condition is nasal inflammatory disease, which can result from allergies. Nasal inflammatory disease can take the form of the growth of polyps within the nasal cavity as well as turbinate hypertrophy. In either case, the nasal passages are constricted and it becomes more difficult for the individual to breathe through his or her nose.

In most situations, initial treatment of nasal polyps and turbinate hypertrophy involves the application of topical corticosteroids to the affected area as well as administration of oral corticosteroids. While such steroids can be highly effective in shrinking polyps and the turbinates, invasive surgery is often required as topical and oral corticosteroid delivery often are insufficient to control the growth of nasal polyps and the swelling of the turbinates. This is unfortunate as, whether the surgery is to remove the polyps or reduce the size of the turbinates, such procedures often must be performed in an operating room under anesthesia. This increases both the costs and the risks of treatment. In addition, the relief such procedures provide is often temporary as it is common for removed nasal polyps to return and, as turbinate reduction does not address the underlying inflammatory pathology, for the turbinates to again expand and constrict the nasal passages. Because of this, it is not unusual for an individual who has had nasal surgery to need the surgery to be performed repeatedly on a periodic basis.

Given the disadvantages associated with surgical treatments, not only in relation to nasal inflammatory disease but also other conditions in which surgery is an option, it can be appreciated that it would be desirable to have alternative treatment options. For example, it would be desirable to be able to deliver a substance, such as a drug, within a target tissue in a manner that is efficacious enough to obviate the need for surgery, or at least delay the need for the surgery and/or reduce the frequency with which the surgery must be performed.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1A is a first perspective view of an embodiment of an implant.

FIG. 1B is a second perspective view of the implant of FIG. 1A.

FIG. 1C is a side view of the implant of FIG. 1A.

FIG. 2 is a perspective view of an embodiment of an implantation device that is configured to implant the implant of FIG. 1 into a target tissue.

FIG. 3 is a side view of multiple implants of the type shown in FIG. 1 arranged in series as they would be inside of a barrel of the implanting device of FIG. 2.

FIG. 4 is a schematic view illustrating implantation of an implant within a nasal polyp of an individual.

FIG. 5A is a first perspective view of another embodiment of an implant.

FIG. 5B is a second perspective view of the implant of FIG. 5A.

FIG. 5C is a side view of the implant of FIG. 5A.

FIG. 6 is a perspective view of an embodiment of an implantation device that is configured to implant the implant of FIG. 5 into a target tissue.

FIG. 7 is a detail perspective view of a distal tip of the implantation device of FIG. 6 shown loaded with the implant of FIG. 5.

### DETAILED DESCRIPTION

As described above, it would be desirable to be able to deliver a substance within a target tissue in a manner that is efficacious enough to obviate the need for surgery, or at least delay the need for the surgery and/or reduce the frequency with which the surgery must be performed. Disclosed herein are systems, devices, and methods that enable such delivery. In some embodiments, a small sharp-tipped implant is configured for implantation within a target (e.g., diseased) tissue. The implant comprises one or more substances, such as one or more drugs, that are to be delivered to the tissue over a period of time. As the implant is provided with a sharp tip, it can pierce tissue and, therefore, can be implanted by simply driving it into the tissue, thereby obviating the need for a needle, trocar, or other sharp delivery device. In some embodiments, the one or more substances are released by the implant over an extended period of time, such as several weeks or months, which can be more efficacious than repeated topical application or injection of such substances. In some embodiments, the implant comprises one or more anchoring elements that prevent the implant from migrating or dislodging. In some embodiments, the implant is bioabsorbable so that there is no need to remove the implant.

In the following disclosure, various specific embodiments are described. It is to be understood that those embodiments are example implementations of the disclosed inventions and that alternative embodiments are possible. Such alternative embodiments include hybrid embodiments that include features from different disclosed embodiments. All such embodiments are intended to fall within the scope of this disclosure.

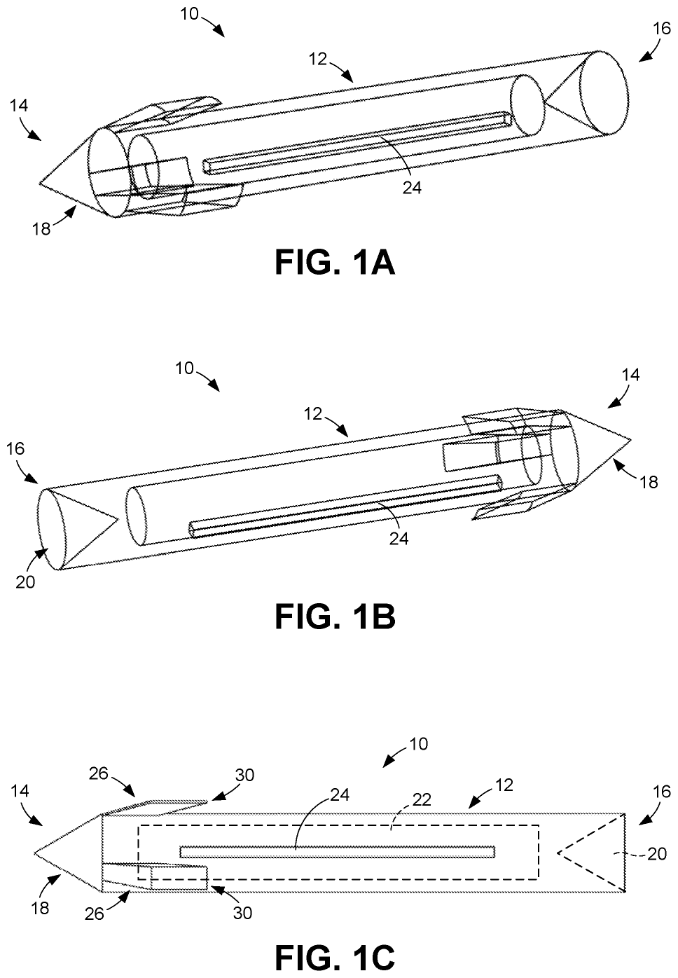

FIGS. 1A-1C illustrate an embodiment of an implant 10 that is configured to deliver a substance within a target tissue in which it is implanted. In some embodiments, the implant 10 is specifically sized and configured for implantation into a nasal polyp or the nasal mucosa from which the polyp extends. Notably, however, that application is only an example as the implant 10, or others similar to it, could be configured for implantation into other tissues of the body.

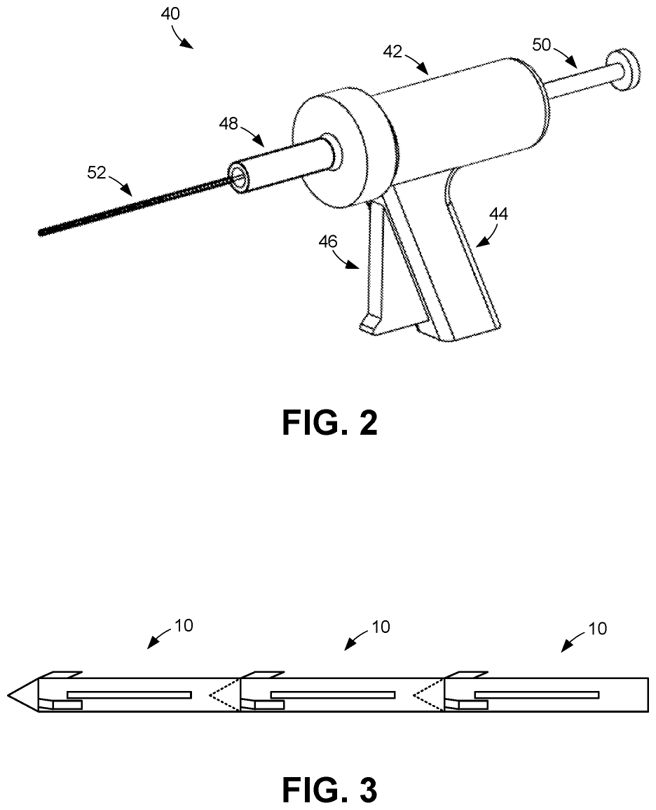

As is shown in FIGS. 1A-1C, the implant 10 generally comprises an elongated body 12. Although the body 12 is illustrated as being generally cylindrical in the figures, it is noted that other shapes are possible as the shape of the body is not critical to its functionality. The body 12 includes a distal (front) end 14 and a proximal (rear) end 16. The distal end 14 forms a sharp, pointed tip 18 that is configured to pierce and cut through target tissue. As is illustrated in the figures, the tip 18 can be generally conical, although any shape that enables the implant 10 to pierce and cut through tissue can be used. When tip 18 comprises a right circular cone, the spread angle of the cone can range from approximately 50 to 70 degrees. As is apparent from FIGS. 1B and 1C, the proximal end 16 of the body includes a cavity 20 that has a size and shape that is specifically configured to receive the pointed tip 18 to enable multiple implants 10 to nest with each other end-to-end, as described below in relation to FIGS. 2 and 3. In this example, in which the pointed tip 18 is generally conical, the cavity 20 defines a generally conical space that the pointed tip of another implant 10 can occupy such that the surfaces of the pointed tip contact the surfaces of the cavity.

Although the size of the implant 10 can depend upon the particular application in which the implant is going to be used, the implant is, in many embodiments, very small. For example, when the implant 10 is configured for implantation into nasal polyps, the body 12 can be approximately 3 to 10 mm (e.g., 6 mm) long and have a cross-sectional dimension (e.g., diameter) of approximately 0.5 to 2 mm (e.g., 1.5 mm).

In some embodiments, the body 12 can be a solid member that is impregnated with one or more substances that are to be delivered to the target tissue. In other embodiments, such as that shown in FIG. 1C, the body 12 can comprise an internal compartment 22 in which the one or more substances can be contained. In the illustrated embodiment, the compartment 22 is also generally elongated and cylindrical just like the body 12. Although only a single continuous compartment 22 is shown in FIG. 1C, it is noted that, in other embodiments, the compartment can be divided into multiple sub-compartments and/or the body 12 can comprise multiple compartments.

When the body 12 includes one or more internal compartments, such as compartment 22, the body can further comprise one or more apertures 24 that extend through the body to the compartment(s) to enable the one or more substances contained therein to be slowly released into the surrounding tissue. In the illustrated embodiment, the implant 10 comprises a single aperture 24 that is configured as a narrow, elongated slit that extends along the length of the internal compartment 22. While a slit is illustrated in the figures, it is noted that the aperture 24 can have other shapes. For example, the body 12 can be provided with one or more circular apertures that extend to the internal compartment 22. Notably, the number, size, and shape of the apertures can be selected to control the rate at which the one or more substances contained within the body 12 are released into the surrounding tissue. When there are multiple compartments or sub-compartments, each can have one or more associated apertures, which can be of different sizes and shapes to individually control the release rate of each substance contained in each compartment/sub-compartment.

Regardless of whether or not the body 12 includes an internal compartment, the body can be made of a biocompatible, bioabsorbable material, such as polyglycolic acid (PGA), polylactic acid (PLA), poly lactic-co-glycolic acid (PLGA), polydimethylsiloxane (PDMS), poly(L-lactide) (PLLA), and Poly-L/D-lactide (PLDLA). In such a case, there is no need to remove the implant 10 after it no longer can deliver any further substance to the tissue. In some embodiments, the material can include proteolytic/fibrolytic enzymes to prevent capsule formation around implant 10. It is noted that, when the body 12 is made of a bioabsorbable material, one or more of the dimensions of the body can be selected to control the timing of the release of the one or more substances. For example, when the body 12 includes the internal compartment 22, the thickness of the walls can be selected to control how long it takes for them to dissolve to the point at which they no longer contain the one or more substances. In cases in which there are multiple compartments and/or sub-compartments that contain different substances, the thicknesses of the walls can be varied in relation to their associated compartments/sub-compartments to individually control the rates of release of the different substances.

A wide variety of substances can be delivered with the implant 10, whether the substances are impregnated into the material of the body 12 or contained within the internal compartment 22 of the body. Examples of such substances include corticosteroids, antihistamines, antimicrobials, chemotherapeutics, biologic agents, monoclonal antibodies, botulinum toxin, desiccating agents, leukotriene receptor modulations, radioactive substances for brachytherapy, antigens for immunotherapy and desensitization, vasoconstrictors, vasodilators and interleukin modifiers. It is also noted that the implant 10 can be coated with one or more substances as well.

With further reference to FIGS. 1A-1C, the implant 10 includes one or more anchoring elements 26 that are configured to prevent migration or dislodging of the implant after it has been implanted. The anchoring elements 26 can be unitarily formed with the body 12 and, therefore, made of the same material as the body. As is apparent from the figures, the anchoring elements 26 extend radially outward from body 12 and are equally spaced from each other about the circumference of the body. In the illustrated embodiment, the implant 10 comprises three such anchoring elements 26, each positioned near the distal end 14 of the body 12. More particularly, the distal-most portions of the anchoring elements 26 are positioned immediately proximal of the pointed tip 18 at the distal end 14 of the body 12.

In some embodiments, each anchoring element 26 is configured as a swept-back, pointed barb having a sharp tip 30 that faces away from the tip 18 of the body 12 toward the proximal end 16 of the body. In some embodiments, each barb 26 can have a length that is approximately 10 to 20 percent of the total length of the implant 10, a height (in the radially outward direction normal to the surface of the body 12) that is approximately 10 to 20 percent of the length of the barb, and a width that is approximately 15 to 25 percent of the length of the barb.

As is most clearly illustrated in FIG. 1C, each barb 26 of the illustrated embodiment generally forms a parallelogram shape when viewed from the side given that the top and bottom edges of the barb are parallel with each other (and the surface of the body 12) and the distal (front) edge and the proximal (rear) edge of the barb are also parallel with each other (and form an acute angle with the surface of the body). Notably, the angle formed between the distal edge and the bottom edge of the barb 26, as well as the angle formed between the proximal edge of the barb and the surface of the body 12, is a small angle that, for example, can range from approximately 10 to 20 degrees. At the distal (front) edge of the barb 26, this small angle facilitates passage of the implant 10 through tissue during implantation. At the proximal (rear) edge of the barb 26, the small angle, in combination with the relatively small height of the barb (again when viewed from the side as in FIG. 1C) can result in the proximal portion of the barb that forms the sharp tip 30 being flexible. In some embodiments, the proximal portion of the barb 26 is compressed inward toward the body 12 during implantation and then extends back outward into the orientation shown in FIG. 1C after the implant 10 has been implanted so that the implant is less likely to migrate backward and possibly dislodge.

FIG. 2 illustrates an embodiment of an implantation device 40 that is configured to implant implants, such as that shown in FIG. 1, into target tissue. As shown in FIG. 2, the implantation device 40 is designed as a hand-held device that includes a body 42, a grip 44 that extends downward from the body, an actuation mechanism that includes a trigger 46 associated with the grip, a coupling element 48 that extends forward from the body, and a delivery rod 50 that extends into the body from a rear of the body. Shown inserted into the coupling element 48 is a removable and replaceable barrel 52 having an inner lumen that is preloaded with multiple (e.g., 5-10) implants 10. The implants 10 are linearly aligned and nested with each other (in the manner illustrated in FIG. 3) within the barrel lumen. That is, the pointed tip 18 of each trailing implant 10 is received within the cavity 20 of the preceding implant 10, as depicted in FIG. 3. When the implants 10 are aligned in this manner, the forces associated with implantation are evenly distributed within the bodies 12 of the implants such that the walls of the implants (when the implants are provided with internal cavities 22) can be made thinner without risking the integrity of the bodies.

In some embodiments, the barrel 52 can comprise internal channels that are formed in the walls of its inner lumen and configured to receive the barbs 26 of the implants 10. In such cases, the inner lumen of the barrel 52 has an inner transverse dimension (e.g., diameter) that is only slightly larger than the outer transverse dimension (e.g., diameter) of the bodies 12 of the implants 10. In some embodiments, the barrel 52 is flexible to facilitate positioning of the tip of the barrel and, therefore, the location at which the next implant 10 can be implanted.

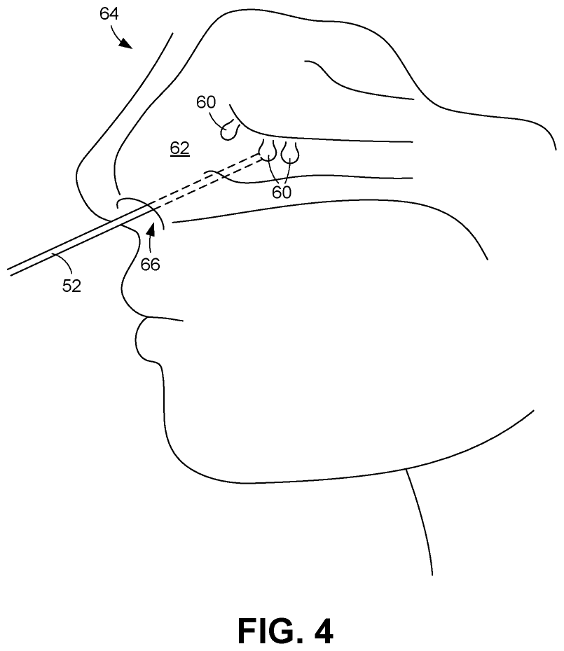

FIG. 4 illustrates an example of implantation of an implant 10 using the implantation device 40. As is apparent from this figure, implants 10 are being implanted within nasal polyps 60 (i.e., intra-polyp implantation) within the nasal cavity 62 of an individual (e.g., patient) 64. To achieve such implantation, the barrel 52 of the implantation device 40 is inserted through one of the patient's nostrils 66 and the distal tip of the barrel is positioned so as to contact a given polyp 60. While FIG. 4 illustrates an example of implantation of an implant 10 within a polyp 60, it is noted that the implant alternatively can be implanted in the nasal mucosa that surrounds the polyp.

Once the tip of the barrel 52 is in position, the actuation mechanism of the implantation device 40 can be activated by an operator (e.g., physician) by pulling the trigger 46 to eject a single implant 10. In some embodiments, ejection of more than one implant 10 is prevented with appropriate means contained within the implantation device 40, such as an indexing mechanism. When the implant 10 is ejected, it is forced into the polyp 60. Specifically, the pointed tip 18 of the implant 10 pierces the polyp 60 so that the implant can lodge within the polyp and the barbs 26 ensure that the implant does not migrate from the position in which it has been implanted. As noted above, inward flexing of the barbs 26 during implantation and the return of the barbs to their original orientations once implantation has been achieved may occur, which prevents the implant from backing out. Further implants 10 can then be implanted into the other polyps 60 and/or surrounding mucosa as desired. Once each implant 10 or each needed implant 10, has been implanted, the barrel 52 can be removed and discarded. A new, preloaded barrel 52 can then be inserted into the coupling element 48 so that the implantation device 40 can be used on the next patient.

Once implanted, the implants 10 release the one or more substances they comprise into the target tissue. As the implant 10 is made of a bioabsorbable material, the implant slowly dissolves within the tissue. In some embodiments, the implant 10 can deliver one or more substances to the tissue for approximately 30 to 90 days, after which time the implant will have been completely dissolved.

Although an implantation device 40 has been disclosed that can be used to implant the implants 10, it is noted that it is possible to implant each implant without using the implantation device. For example, the implants 10 can be implanted using an endoscope. In another example, the implants 10 can be implanted by hand using an appropriate device, such as forceps.

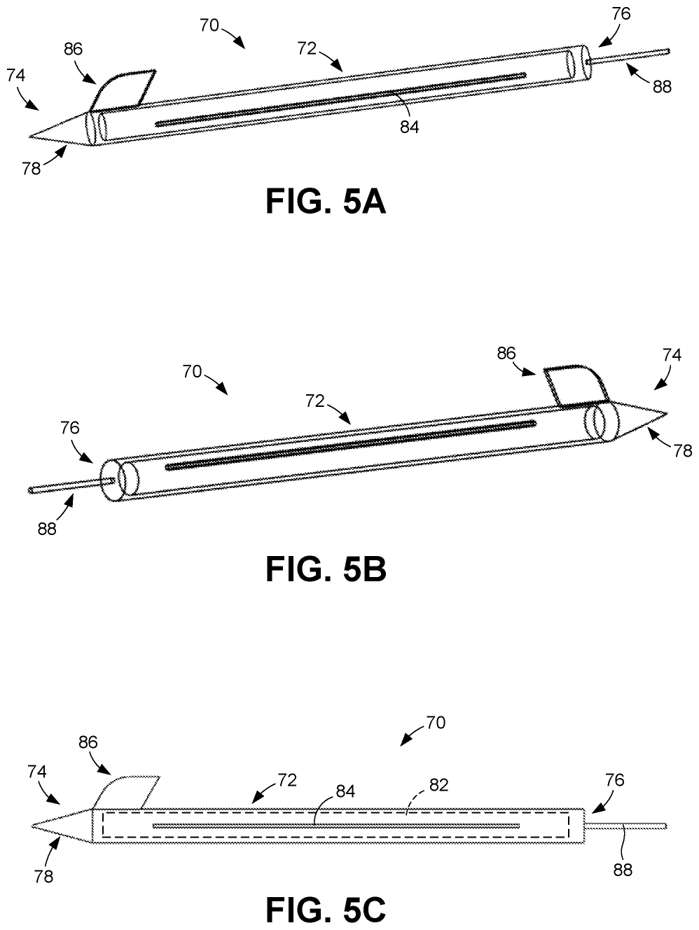

FIGS. 5A-5C illustrate another embodiment of an implant 70 that is configured to deliver a substance within a target tissue. In some embodiments, the implant 70 is specifically sized and configured for implantation into the nasal turbinates. As is shown in the figures, the implant 70 is similar in many ways to the implant 10. Accordingly, the implant 70 generally comprises an elongated body 72 that includes a distal (front) end 74 and a proximal (rear) end 76. The distal end 74 forms a sharp, pointed tip 78 that is configured to pierce and cut through target tissue. As with the tip 18 of the implant 10, the tip 78 can be generally conical. In embodiments in which the tip 78 comprises a right circular cone, the spread angle of the cone can range from approximately 20 to 40 degrees. For the turbinate application, the body 72 can be approximately 5 to 40 mm long and have a cross-sectional dimension (e.g., diameter) of approximately 0.5 to 10 mm.

The body 72 can be a solid member that is impregnated with one or more substances that are to be delivered to the target tissue. Alternatively, as shown in FIG. 5C, the body 72 can comprise an internal compartment 82 (or multiple compartments and/or sub-compartments) in which the one or more substances can be contained. When the body 72 includes one or more internal compartments, such as compartment 82, the body can further comprise one or more apertures 84 that extend through the body to the compartments to enable the one or more substances contained therein to be slowly released into the surrounding tissue. As before, the implant 70 comprises a single aperture 84 that is configured as a narrow, elongated slit, although the number, size, and shape of the apertures can be selected to control the rate at which the one or more substances contained within the body 72 are released into the surrounding tissue.

Regardless of whether or not the body 72 includes an internal compartment, it can be made of a biocompatible, bioabsorbable material, such as one of the materials identified above for the construction of the implant 10. As above, a wide variety of substances can be delivered with the implant 70, whether they are impregnated into the material of the body 72 or contained within the internal compartment 82 of the body.

While the above-described aspects of the implant 70 are similar to those of the implant 10, the implant 70 differs in some respects. First, instead of having multiple anchoring elements, the implant 70 is provided with a single anchoring element 86. The anchoring element 86 is positioned near the distal end 74 of the body 72. More particularly, the distal-most portion of the anchoring element 86 is positioned immediately proximal of the pointed tip 78 at the distal end 74 of the body 72.

In some embodiments, the anchoring element 86 is configured as thin fin that resembles the tail fin of a fish or an airplane. In some embodiments, the fin 86 can have a length that is approximately 5 to 10 percent of the total length of the implant 10, a height (in the radially outward direction normal to the surface of the body 72) that is approximately 40 to 60 percent of the length of the barb, and a width that is approximately 3 to 10 percent of the height of the barb. In such a case and assuming the dimensions identified above for the barbs 26, the fin 86 is both taller and thinner than the barbs of the implant 10 on a relative basis.

As is most clearly illustrated in FIG. 5C, the fin 86 can also have the general shape of a parallelogram when viewed from the side given that the top edge and the bottom edge of the fin are parallel with each other (and the surface of the body 72) and the distal (front) edge and proximal (rear) edge of the fin are parallel with each other (and form an acute angle with the surface of the body). In the case of the fin 86, however, the distal and top edges of the fin form a rounded corner that facilitates insertion of the implant 70 into target tissue.

With reference to FIGS. 5A-5C, the implant 70 further includes a retrieval tab 88 that extends proximally from the proximal end 76 of the body 72. This tab 88 enables the implant 70 to be manually removed with forceps or another surgical device, if desired.

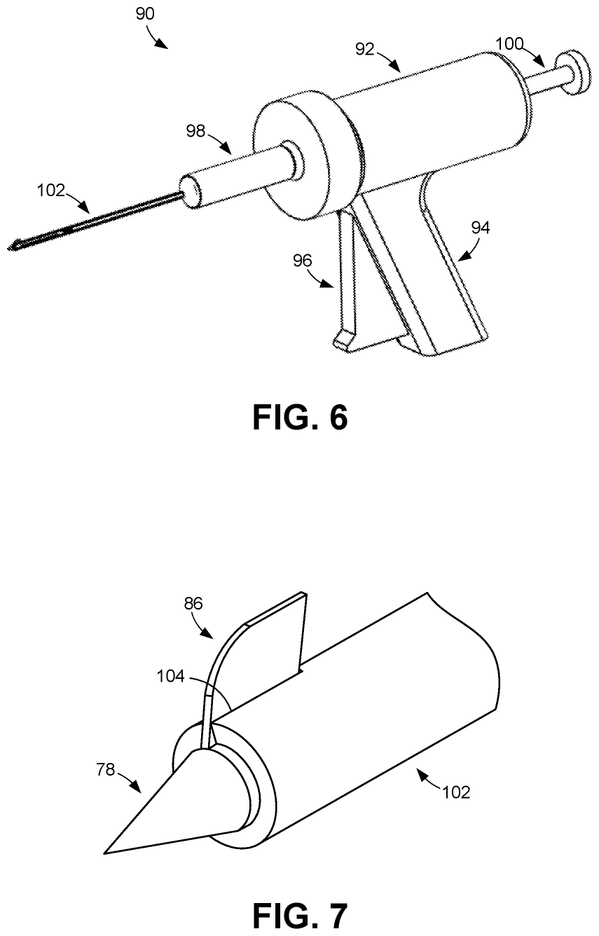

FIG. 6 illustrates an embodiment of an implantation device 90 that is configured to implant implants, such as that shown in FIG. 5. As shown in FIG. 6, the implantation device 90 can have the same configuration of the device 40 shown in FIG. 2. In fact, in some embodiments, the device 90 can be the same device as the device 40. In such an embodiment, a single implantation device 40, 90 can be used to implant the implants 10 as well as the implants 70. Referring to FIG. 6, the implantation device 90 can be a hand-held device that includes a body 92, a grip 94, a trigger 96, a coupling element 98, and a delivery rod 100. Inserted into the coupling element 98 is a removable and replaceable barrel 102 that is specifically configured for use with the implant 70. In cases in which a single implantation device 40, 90 is used for both implants 10 and implants 70, the device is configured to alternately receive both barrels 52 and 102. In other words, the barrels 52, 102 are interchangeable with the implantation device 40, 90. In such cases, an implantation device 40, 90 can be part of an implantation system or kit that further includes a first barrel 52 that is preloaded with multiple implants 10 and a second barrel 102 that is preloaded with one or more implants 70. Referring next to the detail view of FIG. 7, a single implant 70 can be received withing the distal tip of the barrel 102, which includes a slot 104 that is configured to receive the anchoring element 86. In other embodiments, the slot 104 can be longer, in which case the barrel 102 can receive two or more implants 70.

In the case of turbinate implantation, the barrel 102 of the implantation device 90 is inserted through one of the patient's nostrils and the exposed tip 78 of the implant 70 is placed in contact with a turbinate. The trigger 96 of the implantation device 90 can then be pulled by the operator (e.g., physician) to eject the implant 70 into the turbinate. The operator can then rotate the implantation device about the longitudinal axis of the barrel 102 through approximately 90 degrees to fix the implant 70 in place, at which time the barrel can be withdrawn.

As noted above, the disclosed implants and implantation devices can be used in a variety of applications beyond nasal applications. In fact, the implants can be implanted into any soft tissue, cartilage, or bone, as well as polyps, cysts, and tumors, and can be used to deliver substantially any substance to the tissue in which they are implanted. Furthermore, it is noted that the implants can not only be used to deliver substances, they can, in addition or exception, be used to implant devices within a target tissue. Such devices could include electronic devices such as monitoring devices, tracking devices, and even micro- or nanorobotics. In such applications, the implants can be used to implant the electronic devices and the implants can then dissolve, leaving the electronic devices in place within the tissue.

Claimed are:

1. An implant comprising:
a body having a distal end and a proximal end, the distal end including a pointed tip that is configured to pierce and cut through tissue;
an anchoring element extending outward from the body configured to prevent migration of the implant within tissue in which the implant has been implanted; and
a therapeutic substance that is released by the implant into the tissue over time;
wherein the implant is made of a biocompatible and bioabsorbable material.

2. The implant of claim 1, wherein the body is elongated.

3. The implant of claim 1, wherein the pointed tip is conical.

4. The implant of claim 1, wherein the proximal end of the body includes a cavity having a size and shape that is configured to receive the pointed tip of a similarly shaped implant so as to enable linear nesting of multiple implants.

5. The implant of claim 1, wherein the therapeutic substance is impregnated into material from which the body is made.

6. The implant of claim 1, wherein the body includes an internal compartment in which the therapeutic substance is contained.

7. The implant of claim 6, wherein the body further includes an aperture that extends to the internal compartment through which the therapeutic substance is released.

8. The implant of claim 7, wherein the aperture comprises as an elongated slit.

9. The implant of claim 1, wherein the therapeutic substance is a corticosteroid.

10. The implant of claim 1, wherein the therapeutic substance is released over a period of several weeks or months.

11. The implant of claim 1, wherein the anchoring element is positioned near the distal end of the body.

12. The implant of claim 11, wherein the anchoring element comprises a swept-back, pointed barb having a sharp tip that faces toward the proximal end of the body.

13. The implant of claim 12, wherein the implant includes multiple swept-back, pointed barbs each having a sharp tip that faces toward the proximal end of the body.

14. The implant of claim 11, wherein the anchoring element comprises a thin fin.

15. The implant of claim 14, further comprising a retrieval tab that extends proximally from the proximal end of the body configured to enable removal of the implant from tissue in which it is implanted.

16. A delivery system comprising:
an implantation device including a body, a coupling element, and an actuation mechanism;
a first barrel configured to be received by the coupling element of the implantation device, the first barrel containing multiple first implants having pointed tips configured to pierce tissue; and
a second barrel configured to be received by the coupling element of the implantation device, the second barrel containing a second implant having a pointed tip configured to pierce tissue, wherein the second implant has a different size and shape than the first implants;
wherein the actuation mechanism is configured to eject a single implant from a barrel that is received within the coupling element when the actuation mechanism is activated by an operator.

17. A method for treating nasal inflammatory disease of a patient, the method comprising:
driving an implant into swollen nasal tissue of the patient, the implant having pointed tip that enables the implant to pierce the tissue and embed within the tissue without the need for a sharp delivery device, wherein the implant is made of a biocompatible and bioabsorbable material; and
releasing one or more therapeutic substances from the implant into the swollen nasal tissue to shrink the tissue and enable freer breathing through the patient's nose.

18. The method of claim 17, wherein driving an implant into swollen nasal tissue comprises driving the implant into the tissue using an implantation device that ejects the implant from a barrel of the device when a trigger of the device is squeezed.

19. The method of claim 17, wherein releasing one or more therapeutic substances comprise releasing a corticosteroid into the swollen nasal tissue.

20. The method of claim 17, wherein the swollen nasal tissue is a nasal polyp or a swollen turbinate.
